(19) United States
(12) Patent Application Publication
(10) Pub. No.: US 2005/0214945 A1
(43) Pub. Date: Sep. 29, 2005

(54) Htlv-I tax induced killing of p53 null cancer cells

(75) Inventors: Oliver John Semmes IV, Newport News, VA (US); Gary Michael Kupfer, Charlottesville, VA (US)

Correspondence Address: WILMER CUTLER PICKERING HALE AND DORR LLP, THE WILLARD OFFICE BUILDING, 1455 PENNSYLVANIA AVE, NW, WASHINGTON, DC, 20004, US

(73) Assignees: Eastern Virginia Medical School; University of Virginia Patent Foundation

(21) Appl. No.: 10/514,475

(22) PCT Filed: May 16, 2003
(86) PCT No.: PCT/US03/15280

§ 371 Date: May 24, 2005

Related U.S. Application Data
(60) Provisional application No. 60/380,853, filed on May 17, 2002.

(51) Int. Cl. A61K 48/00; C12N 15/867

(52) U.S. Cl. 435/456; 424/93.2; 514/27; 514/34; 514/283; 514/251; 514/449; 514/548; 424/649

(57) ABSTRACT

This invention is based on the finding that expression of an exogenous nucleic acid encoding a polypeptide having human T-cell leukemia virus type I (HTLV-1) Tax activity in p53 null cells results in a sensitization of those cells to DNA damaging agents. Therefore, the present invention is directed to a method of inducing cell death in p53 null cells, enhancing susceptibility to such DNA damaging agents, and the selective cell killing of p53 null cells. It has been found that retroviral vectors, and particularly lentiviral vectors, are suitable for the present invention to permit the transient expression of the HTLV-1 Tax protein in the p53 null cells, which sensitizes the cells.

### RELATED APPLICATION

This application claims priority from U.S. Provisional Application No. 60/380,853, filed May 17, 2002, which is incorporated herein by reference.

### STATEMENT AS TO RIGHTS TO INVENTIONS MADE UNDER FEDERALLY SPONSORED RESEARCH AND DEVELOPMENT

The present invention was made with Government support under grant number CA76595 awarded by the National Institutes of Health. The Government has certain rights in the invention.

### BACKGROUND OF THE INVENTION

Human T-cell leukemia virus type 1 (HTLV-1) is associated with adult T-cell leukemia (ATL) and HTLV-1-associated myelopathy/tropical spastic paraparesis (HAM/TSP) (Gessain, A., et al., Lancet 2: 407-410 (1985); Osame, M., et al., Lancet 1: 1031-1032 (1986); Poiesz, B. J., et al., Proc Natl Acad Sci USA 77:7415-7419 (1980); Yoshida, M., I., et. al., Proc Natl Acad Sci USA 79:2031-2035 (1982)). CD4⁺ T cells are the main target for infection by HTLV-1 and the cellular transformation process is believed to be in large part a consequence of expression of the viral transactivator Tax (Yoshida, M., Annu. Rev. Immunol. 19:475496 (2001)). Tax functions to transactivate viral transcription through interaction with the 5′ Long Terminal Repeat (LTR) (Chen, I., et al., Science 229:54-58 (1985); Felber, G., et al., Science 229:675-679 (1985); Seiki, M., et al., EMBO J. 5:561-565 (1986)). In addition, Tax can activate and/or repress a variety of cellular promoters with potential impact upon cell growth (Franklin, A. A., et. al., J. Biomed. Sci. 2:17-29 (1995); Neuveut, C., et. al., Prog Cell Cycle Res. 4157-62 (2000)). Previously, Tax expression has been shown to reduce cellular genomic stability (Majone, F., et. al., Virology 193(1):456-9 (1993); Semmes, O., et al., Virology 217(1):373-9 (1996)), prompting speculation that induction of genomic instability may facilitate HTLV-1-mediated cellular transformation.

Several studies have attempted to determine how Tax might influence overall cellular genomic integrity. One proffered explanation involves a direct effect of Tax on the transcription of repair gene products, such as β-polymerase (Jeang, K. T., et al., Science 247(4946):1082-4 (1990)) and proliferating cell nuclear antigen (PCNA) (Ressler, S., et al., Journal of Virology 71(2): 1181-90 (1997)). In these cases, Tax repressed or stimulated transcription of the cognate cellular promoter, respectively. Another possible mechanism involves direct interaction of Tax with cellular proteins that monitor/regulate genome integrity (Jin, D. Y., et al., Cell 93(1):81-91 (1998); Majone, F. et al., J Biol Chem. 275(42):32906-10 (2000); Suzuki, T., et al., Virology 270(2):291-8 (2000)). An example of this model is presented by the demonstration that Tax binds to HsMAD 1 (Jin, D. Y., et al., Cell 93(1):81-91 (1998)), thus disturbing spindle assembly/disassembly and progression through M presumably via molecular sequestration. Tax also has both positive and negative effects on cell cycle, each of which may contribute to genomic instability. The positive effects on the cell cycle include activation of kinases and repression of cell cycle inhibitors (Neuveut, C., et al., Mol Cell Biol. 18(6):3620-32 (1998); Lemoine, F. J., et al., J Biol Chem. 276:31851-31857 (2001); Iwanaga, R., et al., Oncogene 20:2055-2067 (2001); Schmitt, I., et al., J Virol. 72(1):633-40 (1998); Santiago, F., et al., J Virol. 73(12):9917-27 (1999); Suzuki, T., et al., Virology 259(2):384-91 (1999)). The negative effects on cell cycle movement could result from a repression of c-myc function (Semmes, O., et al., J Biol Chem. 271(16):9730-8 (1996)), or from activation of cell cycle inhibitors (Yoshida, M., Annu. Rev. Immunol. 19:475-496 (2001)). All of these activities have been ascribed to Tax with a common functional goal as yet undefined. In addition to these various direct effects that may have an impact on genome integrity, Tax may elicit more global effects via its reported activities on the regulatory protein p53.

p53 is an important regulator of cellular genome stability (Lane, D., Nature 358:15-16 (1992)). Induction of p53 following DNA damage can result in activation of repair, cell cycle arrest, and apoptosis (Levin, A. J., Cell 88:323-331 (1997)). In light of the central role of p53 in preserving cellular genomic integrity, it is not unexpected that loss or inactivation of p53 has been causally associated with oncogenic transformation (Donehower, L. A., et al., Biochem Biophys Res Commun. 1155:181-205 (1993); Donehower, L. A., Biochem Biophys Acta. 13:171-176 (1996)). Several recent reports have demonstrated that Tax expression results in inactivation of p53 through several suggested mechanisms. For example, p53 inactivation has been shown to be associated with hyperphosphorylation of serine 15, a residue located in the transcriptional activation domain (TAD) of the protein (Pise Masison, C. A., et al., Mol Cell Biol. 20(10):3377-86 (2000)). In addition, other laboratories have reported that inhibition of p53 function can result from squelching of CREB-binding protein (CBP) by HTLV-1 Tax protein (Suzuki, T., et al., Oncogene 18:41374143 (1999); Van Orden, K., et al., J Biol Chem. 274(37):26321-8 (1999)). In either case, p53 inactivation, which may be expected to impact both DNA repair and clonal survival, could be one of the primary events leading to the clonal expansion of HTLV-1 infected cells.

The above studies have concentrated on a mechanistic understanding of Tax repression of p53 transcription. Several additional studies have suggested an impact of Tax expression on downstream p53 function. Summarizing these data, addition of exogenous p53 can compensate for Tax-induced repair defects (Kao, S. Y., et al., Oncogene 19(18):2240-8 (2000)), and Tax expressing p53 null cells fail to arrest in G1 following addition of exogenous p53 (Mulloy, J. C., et al., J Virol. 72(11):8852-60 (1998)). Clearly it is necessary to determine whether the increased cellular genomic instability of Tax expressing cells arises from impairment of p53 or via p53-independent mechanisms. Although a useful model for some p53-dependent activities, the addition of exogenous p53 does not address the functional intactness of an endogenous p53-mediated response. Furthermore, stable lines expressing Tax likely represent selected/adapted cellular changes to compensate for Tax expression and may not represent direct Tax effects.

In accordance with the present invention, it was observed that p53 is one class of gene products that regulates overgrowth of cells including ensuring the pausing or suicide of cells exposed to DNA damage. p53 carries pleiotropic functions regulating many aspects of cell growth, differentiation, and stress response. As such, nearly half of all cases of cancers involve deletions in or epigenetic repression of the p53 gene. Moreover, deletion of p53 function presents therapeutics with greater difficulty in selective destruction of the host cancer cell. This is primarily due to the increased resistance to apoptosis-inducing stress signals, such as is utilized in chemotherapy and radiation therapy. Numerous therapeutic approaches have centered on restoration of p53 function, induction of p53-independent apoptosis and immunoactivation. However, there is a continuing need to develop approaches to achieve cell death in overgrowing cells in p53-independent cell methodologies.

### SUMMARY OF THE INVENTION

The present invention is directed to a method of reducing viability or inducing cell death of a targeted p53 null cell. The method comprises introducing into the targeted cell a nucleic acid encoding a polypeptide having human T-cell leukemia virus type I (HTLV-1) Tax activity. The polypeptide having HTLV-1 activity, when expressed in an effective amount in the targeted cell, is thereby capable of enhancing the sensitivity of the targeted cell to a DNA damaging agent. Upon being sensitized in this manner, the targeted cell is then contacted with or exposed to a DNA damaging agent which thereby reduces the viability of the cell or induces its death. Preferably, the targeted p53 null cell is a cancer cell.

In accordance with the present invention, the DNA damaging agent can be a chemotherapeutic agent, such as etoposide, adriamycin, amsacrine, actinomycin D, VP16, camptothecin, colchicine, taxol, cisplatinum, vincristine, vinblastine, and methotrexate. Alternatively, the DNA damaging agent can be exposure to irradiation. In such cases, the irradiation may be delivered by exposing the cells to gamma rays, X-rays, directed delivery of radioisotopes, microwaves, or UV radiation.

The cells treated in this manner may or may not be isolated from the patient prior to introduction of the nucleic acid encoding the HTLV-1 Tax polypeptide. If the cells are isolated prior to introduction of the nucleic acid, then the cells can be reintroduced into the patient after it becomes susceptible to the DNA damaging agents based on the expression of the HTLV-1 Tax polypeptide. Alternatively, the nucleic acid encoding the HTLV-1 Tax polypeptide may be directly introduced into the site of a patient wherein the targeted p53 null cells are located.

The method for introducing the nucleic acid encoding the HTLV-1 Tax polypeptide into the targeted cells may be done in any known manner, and preferably, is introduced via a retroviral vector containing the nucleic acid. More preferably, the retroviral vector is a lentiviral vector.

The present invention is further directed to a method for enhancing the susceptibility of a patient to DNA damaging agents comprising introducing into a targeted p53 null cell of the patient a nucleic acid encoding a polypeptide having HTLV-1 Tax activity, expressing the polypeptide in an effective amount in the targeted cell to thereby enhance susceptibility of the targeted cell expressing said polypeptide to a DNA damaging agent, and administering the DNA damaging agent to the patient.

In addition, a method of inactivating a tumor in a patient in need thereof is provided. The method comprises introducing into a patient's tumor cells a nucleic acid encoding a polypeptide having HTLV-1 Tax activity, expressing said polypeptide in an effective amount in the tumor cells, thereby enhancing sensitivity of the tumor to a DNA damaging agent, and contacting the tumor with a DNA damaging agent, wherein the tumor is inactivated.

The present invention further provides a method for the selective killing of p53 null cells. The method comprises introducing into the targeted p53 null cell a nucleic acid encoding a polypeptide having HTLV-1 Tax activity, expressing said polypeptide in an effective amount in the targeted p53 null cells, thereby enhancing sensitivity of those cells to a DNA damaging agent, and administering to the cells a DNA damaging agent, wherein the p53 null cells are selectively killed.

### BRIEF DESCRIPTION OF THE FIGURES

FIG. 1 illustrates the efficiency of viral transduction. (A) shows a depiction of the pHRTaxiGFP transducing vector construct and the control pHRGFP vector. (B) shows REF52 cells transduced by either pHRGFP alone, or bicistronic pHRTaxiGFP. The cell extracts were loaded as follow: pHRGFP (lane 1), pHRTaxiGFP (lane 2), and detected using anti-Tax.

FIG. 2 shows the impaired nucleotide excision repair in Tax-expressing cells. Host cell reactivation assay (HCR) was performed to determine cellular DNA repair activity. (A) depicts the cat reporter plasmid was exposed to different UV doses as indicated. DNA repair activity is reflected in relative recovery of CAT activity and expressed as a percent conversion of chloramphenicol. Normal cells (Normal) are compared to repair deficient Xeroderma pigmentosum-A cells (XP). (B) depicts the repair capacity of Tax expressing cells normalized to non-expressing control cells. Shown is the recovery of repair in Tax-expressing cells as a percent of control. The results are an average of three repetitions.

FIG. 3 depicts UV-induced apoptosis in Tax-expressing cells. Tax-expressing (REF+Tax) and control (REF52 and XPA) cells were subjected to UV irradiation and examined for apoptotic events. Prior to exposure to UV, the percent of non-apoptotic, apoptotic and necrotic cells were examined. In the absence of V, Tax-expressing cells, similar to control REF52 and XP-A cells, showed a low percentage of apoptotic cells (−UV). Following UV exposure both REF52 and REF+Tax displayed a moderate increase of apoptosis. Whereas XP-A cells showed a significantly increased apoptotic response (+UV). These experiments were done in triplicate.

FIG. 4 represents nuclear accumulation, stabilization of p53 and induction of p21 in response to UV-irradiation in Tax-expressing cells. (A) shows REF52 cells transduced with pHRTaxiGFP to 25% efficiency and mounted on coverslips. 48 hours later, the cells were UV-irradiated (20 j/m²), fixed and immunostained with a mouse monoclonal anti-P53 and rabbit polyclonal anti-Tax antibody. Secondary antibodies were anti-mouse FITC-conjugated and anti-rabbit TRITC-conjugated respectively. Shown are separate images of the same field of view which encompasses two cells. The arrows indicate the nucleus of each cell. Both REF52 and REF+Tax cells showed equivalent nuclear accumulation of P53 (left panel). (B) shows REF52 cells transduced with either pHRGFP or pHRTaxiGFP, then exposed to 20 j/m² UV (+) and harvested and subjected to western blot analysis. When probed with anti-P53 antibody, both cell groups demonstrated stabilization of p53 resulting in increased steady-state protein levels. (C) shows the same cells from (B) after they were harvested for 0, 8 and 24 hours. The immunoblots were prepared as described above. The blots were probed with anti-p21 antibody. Shown are extracts from both pHRGFP (−) and pHRTAXiGFP (+) transduced cells.

FIG. 5 represents the failure of Tax-expressing cells to arrest in G1 phase occurs in absence of p53. REF52, REF+Tax, p53d and p53d+Tax cells were synchronized in G0 by serum starvation. The cells were released from G0, exposed to 20 j/m² UV and subjected to FACS analysis for DNA content using propidium iodide as described. The percent of cells occupying G0/G1, S and G2/M were calculated using ModFit software. Shown is the relative distribution of cells at 12 hours following UV exposure.

FIG. 6 depicts increased cell killing in response to UV irradiation in p53 deleted cells. Tax-expressing and control cells were exposed to sublethal doses of UV and examined for percent surviving cells 24 hours after treatment. Tax-expression in a p53+/+ (REF+Tax) or p53 mutant background (HeLa+Tax) showed no decreased percent of surviving cells over the appropriate control cells. However, Tax-expression in the p53−/− background (p53d+Tax) resulted in significant cell death in response to UV exposure.

FIG. 7 illustrates UV-induced apoptosis in p53 deleted cells. p53d and p53d+Tax were placed in asynchronous culture. The cells were UV irradiated at increasing UV doses and accessed for apoptosis. Shown is the percent of cells undergoing apoptosis determined at 0, 12 and 24 hours post irradiation. The doses examined were 0 j/m² (A), 20 j/m² (B) and 50 j/m² (C). These experiments were conducted in triplicate.

FIG. 8 describes plasmids used to generate the construct described in Example 1 and as set forth in Naldini, Science 272:263-267 (1996). This is a schematic representation of the HIV provirus and the three-plasmid expression system used to generate a pseudotyped HIV-based vector by transient transfection. For the HIV provirus, the coding region of viral proteins is shown. The splice donor site (SD) and the packaging signal (φ) are indicated. In the packaging construct, the reading frames of Env and Vpu are blocked (X). In the env-coding plasmid, the coding region of 4070a amphotropic MLV envelope is flanked by a MLV LTR and a SV40 poly(A) site. The VSV G coding region is flanked by the CMV promoter and a poly(A) site. In the transfer vector, pHR′, the gag gene is truncated and out of frame (X), and the internal promoter CMV is used to drive expression of either β-galactosidase (lacZ) or luciferase cDNA. The Rev responsive element (RRE) and splice acceptor site (SA) are shown.

### DETAILED DESCRIPTION OF THE INVENTION

The present invention is directed to a method of reducing viability or inducing cell death in targeted cells. The method involves rendering targeted cells sensitive to DNA damaging agents by introducing into the targeted cells a nucleic acid encoding a polypeptide having HTLV-1 Tax activity. In accordance with the present invention, it has been found that Tax alone induces a state of genomic instability in target cells, and particularly p53 null cells, to render the tax-expressing cells incapable of normal cellular damage-repair response through bypass of the appropriate cell cycle checkpoint nucleotide repair system. When such cells are then exposed to a DNA damaging agent, cell death is induced. Preferably, the targeted cells are p53 null cells.

As used herein, the “targeted cells” of the present invention are those cells selected for induction of cell death and characterized by inappropriate cell proliferation. Any cell or cell type may be used, but preferably the cell or cell type is that associated with degenerative disorders, including cancers, such as carcinomas such as adenocarcinomas, squamous carcinomas, carcinoma of the organs including breast, bladder, colon, head, neck, etc.; sarcomas including chondrosarcoma, melanosarcoma, etc.; and leukemia and lymphomas including acute lymphomatic leukemia, acute myelogenous leukemia, non-Hodgkin's lymphoma, Burkitt's lymphoma, B-cell lymphomas, T-cell lymphomas, etc., and autoimmune disorders. Preferably, the targeted cells include host cancer cells or tumor cells and may include lymphocytes, including T-cells, fibroblasts, epithelial cells, endothelial cells, and keratinocytes. Preferably, the targeted cell is a p53 null cell. As used herein, “p53 null cell” refers to a cell that does not express p53 or lacks p53 activity. For example, some known human cancer p53 null cells include HT1080 fibrosarcoma (Anderson et al., Genes, Chromosomes & Cancer 9:266-281 (1994), Saos2 osteosarcoma (Subler and Martin, J. of Virology 68:103-110 (1994)), NCI-H23 non-small cell lung carcinoma (Takahashi et al., Cancer Research 52:2340-2343 (1992)), and RD rhabdomyosarcoma (Felix et al., Cancer Research 52:2243-2247 (1992)). However, the p53 activity or expression in cells isolated or targeted for insertion of the tax nucleic acid in accordance with the present invention may be assayed according to known methods. The targeted cells are isolated from mammals, preferably human, rat, or mouse.

It has further been found that the targeted cells have increased cell death (reduced viability) upon de novo expression of Tax by the transformed cell and subsequent exposure to DNA damaging agents. “De novo expression” of Tax refers to the lack of expression of Tax in the targeted cell prior to transformation. For example, the use of naive cells or a first generation population of cells isolated from a source in which the genome has not been genetically altered, such as by stable insertion or otherwise transformed, in which the Tax protein is not expressed, may be used in accordance with the present invention. Thus, it has been found that the de novo expression of Tax reflected early cellular responses to Tax expression, whereas events occurring in long-term or stable Tax-expressing systems could represent adaptations of the cell to Tax expression and may reflect long-term Tax effects.

Similarly, the expression of Tax in the transformed targeted cell may be limited in time or transient. “Transient expression” indicates that the transformed cell expresses the gene product encoded by the inserted nucleic acid for short periods of time or only during proliferation and expansion of the cells.

The targeted cells are transformed with a vector comprising a nucleic acid molecule encoding a polypeptide having HTLV-1 Tax activity. Tax is a known protein and the nucleic acid molecules encoding Tax have been previously disclosed (Seiki et al., Science 228:1532-1534 (1985)) For example, the amino acid sequence for Tax (Accession No. S67443) and the nucleotide sequence encoding Tax (Accession No. GI 45570) as described in Major et al., J. Gen. Virol. 74 (Pt 11), 2531-2537 (1993) is publicly available in GenBank. However, the present invention is not limited herein to the Tax protein and nucleic acid disclosed in Major et al. One having skill in the art would be capable of selecting a suitable nucleic acid encoding Tax for use in the present invention. As used herein, the “nucleic acid,” “nucleic acid molecule,” or “polynucleotide” refers to a single- or double-stranded DNA, genomic DNA, cDNA, RNA, DNA-RNA hybrid, or nucleotide polymer. The nucleic acid encoding the Tax protein provided by this invention can be assembled from cDNA fragments and short oligonucleotide linkers, or from a series of oligonucleotides, to provide a synthetic gene which is capable of being inserted in a recombinant expression vector and expressed in a recombinant transcriptional unit.

In accordance with the present invention, a “polypeptide having HTLV-1 Tax activity” refers a polypeptide having the HILV-1 Tax ability to render the targeted cells expressing the polypeptide sensitive to DNA damaging agents. Preferably, the encoded polypeptide is the HILV-1 Tax protein. However, it is noted that the present invention provides for the introduction of a nucleic acid encoding polypeptides derived from HTLV-1 Tax that sensitive p53 null cells to DNA damaging agents when expressed therein. Notably, this sensitizing of the targeted cells is highly selective for p53 null cells.

Preferably, the tax nucleic acid is present in a suitable expression vector. The term “expression vector” refers to a plasmid, virus or other vehicle known in the art that has been manipulated by insertion or incorporation of the tax nucleic acid. Polynucleotide sequences, which encode Tax, should be operatively linked to expression control sequences. “Operatively linked” refers to a juxtaposition wherein the components so described are in a relationship permitting them to function in their intended manner. An expression control sequence operatively linked to a coding sequence is ligated such that expression of the coding sequence is achieved under conditions compatible with the expression control sequences. As used herein, the term “expression control sequences” refers to nucleic acid sequences that regulate the expression of a nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (i.e., ATG) in front of a protein-encoding gene, splicing signal for introns, maintenance of the correct reading frame of that gene to permit proper translation of mRNA, and stop codons. The term “control sequences” is intended to included, at a minimum, components whose presence can influence expression, and can also include additional components whose presence is advantageous, for example, leader sequences and fusion partner sequences. Expression control sequences can include a promoter.

By “promoter” is meant the minimal sequence sufficient to direct transcription. Also included in the invention are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters, are included in the invention (see e.g., Bitter et al., Methods in Enzymology 153:516-544 (1987)). When cloning in mammalian cell systems, promoters derived from the genome of mammalian cells (e.g., metallothionein or elongation factor-1 alpha promoter) or from mammalian viruses (e.g., the retrovirus long terminal repeat; the adenovirus late promoter; the vaccinia virus 7.5K promoter; the cytomegalovirus promoter; the Rous Sarcoma virus promoter; the Moloney Sarcoma virus promoter) may be used. Promoters produced by recombinant DNA or synthetic techniques may also be used to provide for transcription of the nucleic acid sequences of the invention.

Moreover, useful expression vectors can further comprise a selectable marker that may be used to ascertain successful incorporation of the intended nucleic acid. Suitable selectable markers include green fluorescent protein, antibiotic resistance, such as for ampicillin and tetracycline resistance, neomycin, zeocin, hygromycin, and recessive markers such as thymidine kinase (TK), dihydrofolate reductase (DHFR), adenine phosphoribosyl transferase (APRT) and hypoxanthine phosphoribosyl transferase; thus providing a simple means for identifying transformed cells.

The vectors are preferably constructed to obtain the transient expression of the introduced sequence. For example, the tax nucleic acid may be operably linked to a promoter for introduction into a targeted cells as a non-replicating DNA (or RNA) molecule, which may either be a linear molecule or a closed covalent circular molecule which is incapable of autonomous replication. In such case, transient expression of the Tax protein by the cell may occur.

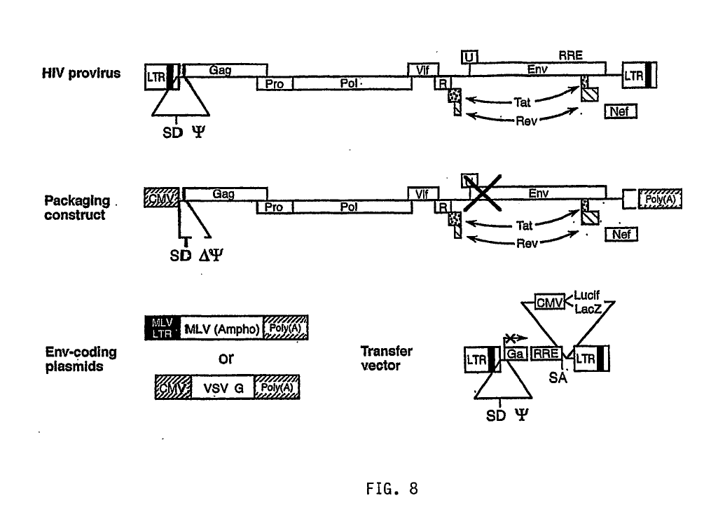

In a preferred embodiment, the packaging construct used in the present invention is an HIV-based plasmid in which the LTR is replaced with the human cytomegalovirus promoter, which drives the expression of all the viral proteins except vpu and env products. In this construct, the packaging signals are removed so that this construct's RNA is not packaged. The gene-transferring vector contains all of the sequences required for RNA packaging and reverse transcription. FIG. 8 illustrates the transducing plasmid vectors. The transfer vector may be rev and tat dependent. The third plasmid supplies the pseudotyped VSV-G expressed under the control of the CMV promoter. Moreover, an internal ribosomal entry site (IRES) sequence has been introduced so that Tax and GFP can be co-expressed, thus allowing for rapid selection of living Tax-expressing cells as shown in FIG. 8D. Additionally, FIG. 8E describes yet another construct within the scope of the present invention comprising a double IRES vector capable of expressing 3 separate gene products. This gene delivery system results in a very high percentage of infected cells and is capable of infecting non-proliferating cells. Preferably, the construct pHRTaxiGFP comprises produced packagable RNA, which can be used to cotransfect mammalian cells by the calcium phosphate method to produce replication-defective viral particles.

As used herein, a cell is transformed with the tax nucleic acid by introducing or inserting the nucleic acid into the targeted cell. “Introducing” the nucleic acid encompasses any method of inserting an exogenous nucleic acid molecule into a cell and includes, but is not limited to, transduction, transfections, microinjection, and viral infection of the targeted host cells. Ideally, the choice of a gene delivery system will be made by those of skill in the art, keeping in mind the objectives of efficient gene transfer, with an appropriate level of gene expression, in a cell-specific manner, and without any adverse effects. For example, transforming a mammalian cell with DNA encoding the HTLV-I tax protein can be accomplished using many different vector systems, depending upon whether it is desired to insert the Tax DNA construct into the host cell chromosomal DNA, or to allow it to exist in an extrachromosomal form.

A preferred manner for introducing the HILV-I Tax encoding nucleotide sequences (and their functional equivalents and/or hybrids and/or mutants) is by the use of viral vectors. Suitable viral vectors for gene transfer include retroviruses (Miller et al., Methods Enzymol. 217:581-599 (1993)) including human immunodeficiency virus (HIV), adenovirus derivatives (Erzurum et al., Nucleic Acids Res. 21:1607-12 (1993); Zabner, et al., Nat. Genet. 6:75-83 (1994); Davidson, et al., Nat. Genet. 3:219-223 (1993)) adeno-associated virus (AAV), (Flotte, et al., Proc. Natl. Acad. Sci. 90:10613-7 (1993)) and Herpes virus vectors (Anderson, et al., Cell Mol. Neurobiol. 13:503-15 (1993)). Other suitable viruses can be readily selected and employed by those of ordinary skill in the art. Other methods for DNA delivery include liposome mediated gene transfer (Alton, et al., Nat. Genet. 5:135-42 (1993); Nabel, et al., Proc. Natl. Acad. Sci USA 90:11307-11 (1993)). The use of viral vectors for introduction of genes into mammalian cells is also reviewed, for example, in Varmus, Science 240(4858):1427 (1988); Eglitis et al., BioTechniques 6,7:608 (1988); Jaenisch, Science 240(4858):1468 (1988); and Bernstein et al., Genet. Eng. (N.Y.) 7:235 (1985).

Retroviral vectors can be used to transfer genes efficiently into the targeted cells by exploiting the viral infectious process. Foreign or heterologous genes cloned or inserted into the retroviral genome can be delivered efficiently to the targeted host cells, which are susceptible to infection by the retrovirus. Through well-known genetic manipulations, the replicative capacity of the retroviral genome can be destroyed. The resulting replication-defective vectors can be used to introduce the tax nucleic acid to the targeted cell, but the virus would not be capable of replicating. In addition, a helper virus or packaging cell line can be used to permit vector particle assembly and egress from the cell. A “vector particle” or “retroviral particle” refers to the viral-like particles that are capable of introducing nucleic acids into a cell through a viral-like mechanism. In accordance with the present invention, any retroviral vector capable of inserting the nucleic acid into the targeted host cell can be used in the present invention. For example, the amphotropic Moloney murine leukemia (MoMLV), vesicular stomatitis virus G-glycoprotein (VSV-G) pseudotyped replication-defective lentiviral (Naldini, L., et al., Science 272:263-267 (1996)), or any other selective or non-selective viral vectors for gene delivery. Preferably, a lentiviral transduction system is used.

Once the tax nucleotide sequence is introduced into the targeted cell and expressed therein, the expression of the Tax protein renders the transformed cell hypersensitive to DNA damaging agents. DNA damaging agents are well known in the art and include, for example, chemotherapeutic agents and irradiation. Chemotherapeutic agents are chemical agents or drugs used in chemotherapy treatment which selectively affects tumor cells and includes etoposide, adriamycine, amsacrine, actinomycin D, VP16, camptothecin, colchicines, taxol, cisplatinum, viscristine, vinblastine, and methotrexate. In addition, irradiation means exposing the cell, tissue or organ to photons, electrons, neutrons or other ionizing radiations and include gamma rays, X-rays, directed delivery of radioisotopes, microwaves, and UV radiation. The sensitized cell is “contacted” or “exposed” to the DNA damaging agent by delivery of the chemotherapeutic agent directly or near to the cells or by exposure of the sensitized cell to the irradiation, as is well known in the art. To achieve the cell killing, the DNA damaging agent is delivered in an amount sufficient to selectively kill the targeted cells.

The “selective killing” of the targeted cell refers to the unexpected finding that p53 null cells transformed with the tax nucleic acid are preferentially sensitized to DNA damaging agents relative to p53+ cells or normal cells. In accordance with the present invention, the transformed p53 null cells exposed to a chemotherapeutic agent or irradiation exhibits a cell death percentage of at least about 50%, preferably at least about 60%, more preferably at least about 70%, and most preferably at least about 80-100%. Notably, the control cells (p53+ cells) exhibit a cell death percentage upon exposure to chemotherapeutic agents or irradiation in an amount of about 2-10%. Therefore, it is seen that Tax expression in p53 null cells causes the surprising result of a high percentage of cell death in these cells as compared to control cells.

One having ordinary skill in the art would be capable of ascertaining the amount of cell death. For example, a dose response assay to assess cell viability or agarose gel electrophoresis of DNA extractions to determine DNA fragmentation, a characteristic of cell death, may be used to quantify the amount of cell death. In addition, other assays, such as a chromatin assay, or drug resistance assays (Lowe et al., Cell 74:957-967 (1993)) may also be used to determine the effect of Tax on the sensitizing transformed targeted cells and their response to Tax and chemotherapy agents or irradiation.

A patient may be treated in accordance with the present invention by the introduction of the tax nucleic acid into the targeted cells, which may be first isolated from the patient. In this manner, a population of the targeted cells is isolated according to known methods in the art and the tax nucleic acid is introduced into the targeted cells. The targeted cells express de novo the Tax protein, which renders the Tax-expressing cells sensitive to cell death. These cells are transferred back into the patient and the patient then undergoes chemotherapeutic treatment or irradiation to induce cell death.

In yet another method, the tax nucleic acid may be directly introduced into the tumor site, which would then get incorporated into the targeted cells. The transformed targeted cells would then be increasingly sensitive to the chemotherapeutic treatment or irradiation upon administration. Thus, the invention is further directed to a method for enhancing the response to chemotherapeutic treatment or irradiation.

The present invention further includes a method for treating cells, tissues or organs in which the p53 mediated function is relevant to cell, tissue or organ state, including but not limited to inappropriate cell proliferation or inappropriate cell persistence.

Reference will now he made to specific examples illustrating the compositions and methods above. It is to be understood that the examples are provided to illustrate preferred embodiments and that no limitation to the scope of the invention is intended thereby.

### EXAMPLES

### Example 1

Materials

The plasmids used for retroviral transduction were made as previously described in Naldini, L., et al., Science 272:263-267 (1996) and U.S. Pat. Nos. 6,428,953 and 6,555,342, herein incorporated by reference. pMD.G was used for the production of the envelope protein G of vesicular stomatitis virus. PCMV(delta)8.2, was the packaging construct, and was used for the production of Human Immunodeficiency Virus gag, pol and regions of env. The delivery construct pHRTax was made by inserting the tax ORF (GenBank No. S67443; Accession No. GI 455730), into the Xho I and Bgl II site of pHRCMV and produced “packagable” viral RNA. pHRTaxiGFP and pHRGFP produced either Tax-GFP and GFP packagable RNA. pRSV-CAT contained the cat (chloramphenicol acetyltransferase) reporter gene under the control of the RSV (Rous Sarcoma Virus) promoter, and pMSV-Luc contained the luciferase gene under the control of MSV (Moloney Sarcoma Virus) promoter. The REF52 (Rat Embryonic Fibroblasts) cell line was provided as a gift from Thomas Parson (University of Virginia), the p53−/− cell line was a gift from Bert Vogelstein (Johns Hopkins University), and the XP-A (Xeroderma pigmentosum complementation group A) cell line GM04429 was obtained from Coriell Cell Repositories (NIGMS). The cells were maintained at 37° C. in Iscove's Modified Dulbecco's Medium with 10% Fetal Calf Serum and 1% Penicillin-Streptomycin (GibcoBRL). The anti-Tax rabbit polyclonal antibody was raised against amino acids 104 to 120 of the Tax protein (corresponding to GI 455730; Accession No. AAP14011) and was affinity purified with the same peptide. Antibodies against p53 (DO-1) and p21 (F-5) were purchased from Santa Cruz Biotechnology. Anti-BrdU (BU-3) was purchased from Sigma.

Immunoblot Analysis

Approximately 2×10⁶ cells were harvested and proteins extracted with 200 μM-Per Mammalian Protein Extraction Reagent (Pierce). 50 μl of 4× Laemmli buffer was added to the lysate. 301 μl of the lysate was electrophoresed through a 10% SDS polyacrylamide gel. The proteins were electroblotted onto an Immobolin-P membrane (Millipore), and probed with the indicated primary antibody and the appropriate secondary alkaline phosphatase-conjugated antibody. Immunoreactivity was detected via Western Star chemiluminescence protein detection (Tropix).

Host Cell Reactivation Assay

pSV2-CAT reporter plasmid was damaged ex vivo by exposure to 1000 j/m² of Uv-C light using a UV chamber-GS Gene Linker (Bio-Rad). REF52 and XP-A cells were transfected with 4 μg of UV-irradiated or non-irradiated pSV2-CAT plasmid together with an undamaged reporter plasmid (pMSV-Luc), and with or without Tax plasmid. Forty-eight hours after calcium phosphate transfection, cells were pelleted and resuspended in 250 μl of 250 mM Tris pH8.0. For the Luciferase assay, 25 μl of the total cellular extract was added to 50 μl of luciferase substrate. Luciferase activity was quantitated in a Luminometer. Cat assays were performed in parallel with the same cells as described (Semmes, O., et al., J. Virol. 66(12):7183-92 (1992)). Cat activity was normalized to luciferase activity of the same extract. Repair activity was calculated by setting normalized cat activity from cells cotransfected with non-irradiated pSV2-CAT to 100%. The repair activity of duplicate cells cotransfected with irradiated pSV2-CAT was reported as a percentage of that activity.

Global Nucleotide Excision Repair Assay

REF52 cells were seeded onto glass coverslips and transduced to express Tax at 25% efficiency. Sub-confluent Tax-expressing REF52 cells were cultured in 0.5% serum for 48 hrs, to synchronize at G0, prior to irradiation. The cells were released from G0 with addition of complete medium. At four hours post-release, the cells were irradiated with UV light (20 j/m²) using a UV chamber-GS Gene Linker (Bio-Rad) and incubated for 30 min with 10 μM BrdU containing medium. The cells were then washed 4× with PBS and fixed in 4% paraformaldehyde. Cells were permeabilized with 2 minutes incubation in 100% methanol and washed 4× with PBS. The prepared cells were then reacted with both anti-Tax and anti-BrdU in PBS (containing 2% BSA). Primary antibodies were removed by washing 4× with 1% Tween 20 in PBS (PBS-Tween). Secondary conjugated antibodies were reacted for one hour at room temperature (RT). The cells were then washed with PBS-Tween and the slips were inverted onto slides with Vecta Shield (Vector Laboratories, CA). Incorporation of BrdU into the nucleus of cells at G1 corresponded to unscheduled repair synthesis.

Viral Transduction

The lentiviral transduction system as described by Naldini et al. (Naldini, L., et al., Science 272:263-267(1996)) was used. Three plasmids (pHRTaxiGFP orpHRGFP produced packagable RNA; pCMVΔ8.2 produced gag, pol and accessory gene products; and pMD.G produced VSV G protein) were cotransfected into 293/T17, by the calcium phosphate method, to produce replication-defective viral particles. Viral titre was determined as relative to control Green Fluorescent Protein (GFP) producing virus stock. The expression of GFP was assessed as percent of green fluorescent cells. The standard curve of p24 values associated with increasing expression efficiencies was used as an estimate of potential infectious units. p24 values were derived for each batch of virus supernatant.

Target cells were plated at 2-3×10⁵ cells per mil in serum-free medium. Supernatant containing lentiviral vector particles were added at a concentration corresponding to 1×10⁶ infectious units per ml. Cells were incubated for 24 hours then washed and cultured in vitro for 48 hours to ensure maximal transgene expression. GFP expression was analyzed in target cells by fluorescent microscopy.

Apoptosis Studies

Tax-expressing cells were treated with different UV doses (0, 20 and 50 j/m²) and assessed at different time points post-transduction (4, 8, 12 and 24 hrs). Cells were separated into living, necrotic or apoptotic populations using the triple source fluorescent labeling, Vybrant Apoptosis Assay kit (Molecular Probes) according to the manufacturer's recommendations.

Cytokinesis Block Cell Cycle Progression Assay

Asynchronous cell cultures were seeded on coverslips and exposed to UV irradiation and allowed to recover for 1 hour. Cytochalasin B was added and the cultures incubated for 36 hours. The coverslips were fixed with paraformaldehyde/methanol and stained with propidium iodide. Slides were prepared and cell nuclei examined by microscopy. Bi-nucleus cells were considered dividing.

Flow Cytometry

For cell cycle analysis, cells were collected by gentle scraping following a rapid EDTA rinse and concentrated by low speed centrifugation and washed in 1×PBS, and fixed with cold 70% ethanol. Cells were stained with a PI solution (PBS 1×, RNase A [10 μg/ml], and Propidium Iodide [50 μg/ml]) followed by cell sorting analysis. FACS data acquired were analyzed by CellQuest software.

Viral Transduction Results in Efficient De Novo Expression of Tax

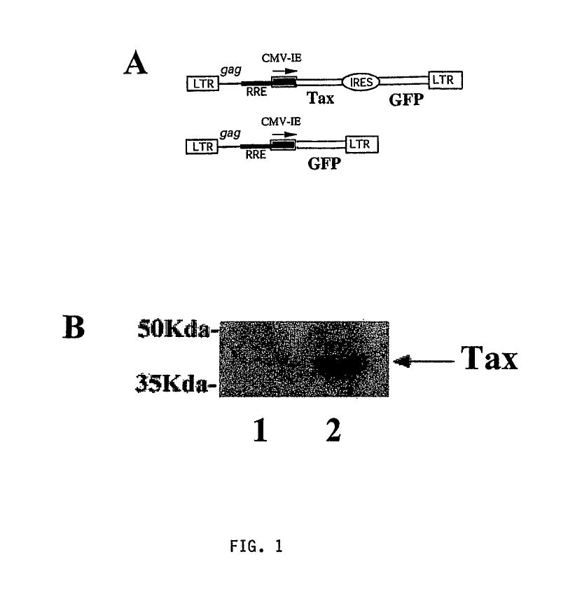

To test the efficiency of the viral transduction for de novo expression of Tax, REF52 cells were transduced to express either Tax and GFP or GFP alone. The pHRTaxiGFP vector used for delivery of the bicistronic message expressing Tax and GFP and the corresponding control vector expressing GFP alone are shown in FIG. 1A. The transduction efficiency of this system was determined as a measure of the resulting expression of the delivered gene. Tax-expressing REF52 cells were identified via their bi-cistronic expression of both Tax and GFP. Titration of the viral supernatant to a level resulting in 50% of cells expressing GFP resulted in endpoint measurement of transduction titre. By this definition, typical viral titres were 1×10⁶ infectious units per ml without supernatant concentration. The expression of Tax in the target cells was verified by Western blotting. FIG. 1B shows the expression of Tax following transduction of the pHRTaxiGFP cDNA using anti-Tax antibody. Thus efficient production of Tax and GFP were achieved and can be quantitated using the described viral-mediated transduction method.

HTLV-1 Tax-Expressing Cells Display an Impaired Nucleotide Excision Repair

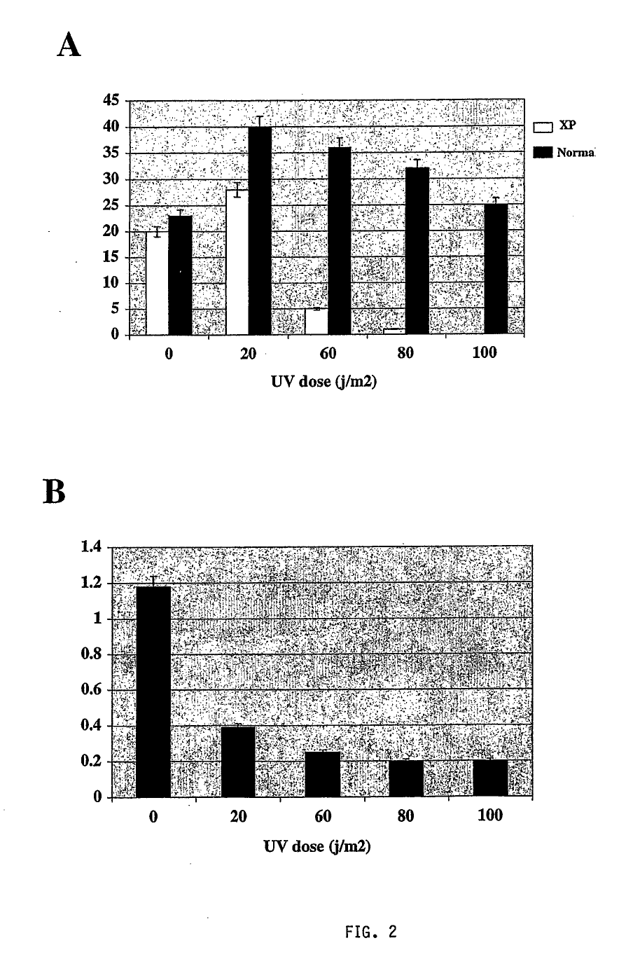

In examining the biological response of Tax-expressing cells to UV-induced DNA damage, efficient Nucleotide Excision Repair (NER) ability was tested. The Host Cell Reactivation Assay (HCRA) was used as a measure of NER capacity in Tax-expressing REF52 cells. As a control, the differential repair competency of the Xeroderma Pigmentosum complementation group-A (XP-A) cell line GM04429 and the non-XP cell GM00010B of the same lineage was examined. When examined for NER capacity in the HCRA, the XP-A cell line showed significantly reduced repair capacity when compared to the non-XPA cell GM00010B (FIG. 2A). When Tax-expressing REF52 cells were compared to control GFP-expressing REF52 the Tax-expressing cells were significantly reduced in NER capacity (FIG. 2B). Thus, Tax expression results in reduced NER capacity as measured by HCRA.

Cell Cycle Arrest and Induction of Apoptosis in HTLV-1 Tax-Expressing Cells

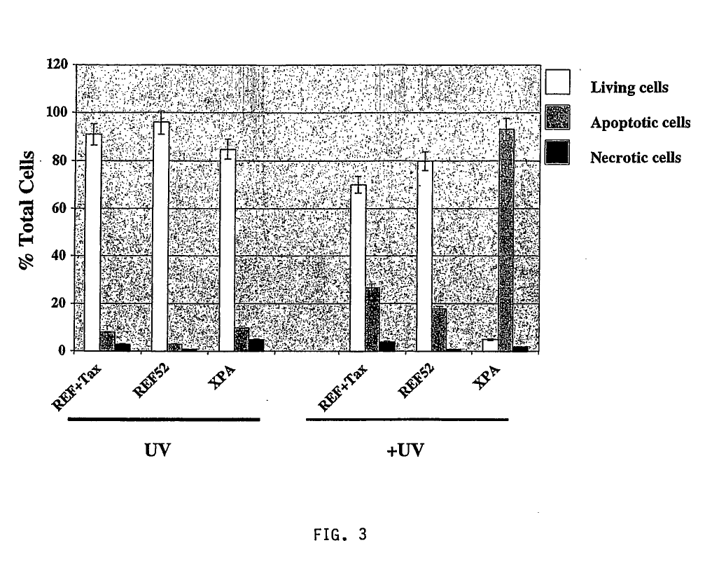

In addition to repair, at least two key events occured in the UV-induced cellular DNA damage response. These are initiation of cell cycle arrest and induction of apoptosis. The ability of Tax-expressing REF52 cells to undergo cell cycle arrest and apoptosis was assessed. Tax-expressing and control cells were subjected to UV irradiation and examined for apoptotic events and cell cycle profile. Prior to exposure to UV, the percent of non-apoptotic, apoptotic and necrotic cells were examined. The three populations were identified using the Vybrant assay. In the absence of UV, Tax-expressing cells, similar to control REF52 and XP-A cells, showed a low percentage of apoptotic cells (FIG. 3; −UV). Following UV exposure both REF52 and Tax-expressing REF52 displayed a normal apoptotic response to DNA damage as indicated by the moderate increase of apoptosis. The same results were seen using several Tax-expressing cells including GM00010B (data not shown). However, the NER repair-deficient XP-A cells showed a significantly increased apoptotic response as was expected (FIG. 3; +UV). Thus, Tax-expressing cells showed a normal apoptotic response, which is vastly more conservative than would be expected from NER-deficient cells.

Next, the cell cycle response of Tax-expressing cells was measured. REF52 and REF+Tax cells were subjected to the cytokinesis block cell cycle arrest assay to determine the intactness of the damage-induced checkpoint. In this assay, asynchronous cells were exposed to UV and incubated with cytochalasin B one hour later. The cell population was examined 36 hours later and assessed for division events. Those cells that failed to arrest appeared as bi-nucleated cells, whereas those that arrest prior to M will appear as mononuclear cells. As expected, cells which were not exposed to UV light continued to divide and resulted in greater than 98% binucleated cells. In contrast REF52 cells exposed to UV light initiated cell cycle arrest and presented a predominately (>90%) mononuclear cell population. However, Tax-expressing REF52 cells, exposed to UV, presented nearly 75% of the population as binucleated cells indicating a failure to arrest in cell cycle in response to UV-induced DNA damage. For each data point, 1000 cells were analyzed. Tax-expressing cells unexposed to UV exhibited the same profile as in the absence of Tax, showing binucleated cells. In total, these results suggested that Tax-expressing cells have an intact early apoptotic response but fail to arrest in cell cycle.

Global Nucleotide Excision Repair of Tax-Expressing Cells is Normal in G1-Synchronized Cells

A failure in damage-induced cell cycle arrest would result in reduced repair efficiency and could explain the Tax-associated NER deficiency. This would imply that the actual NER pathway in Tax-expressing cells is intact and would predict that restoration of a competent cell cycle arrest would restore NER capacity. To test this hypothesis, global NER in G1 synchronized cells was examined. Tax-expressing REF52 cells inter-mixed with REF52 were accumulated in G0 by serum starvation for 72 hours. The cells were then released from G0 and allowed to advance for 4 hours into G1. The cells were then exposed to UV and incubated with BrdU-dUTP. Incorporation of BrdU is restricted to cells undergoing DNA synthesis, which is unscheduled during G1. The cells were then fixed and immunostained with anti-Tax rabbit polyclonal and anti-BrdU mouse monoclonal antibodies. Secondary antibodies were anti-rabbit Texas red and anti-mouse FITC-conjugated respectively. The unscheduled DNA synthesis directly related to NER mediated repair synthesis in UV exposed cells. Under these conditions, the UV-induced unscheduled DNA synthesis in Tax-expressing cells was indistinguishable from control cells. Both REF52 and REF+Tax were actively undergoing unscheduled DNA synthesis following UV irradiation. Those cells unexposed to UV did not incorporate BrdU and demonstrated the successful synchronization out of S phase. No unscheduled DNA synthesis was observed in the absence of UV for either REF52 or REF+Tax. The transduction efficiency was titred to 25% so that REF52 and REF+Tax cells could be viewed adjacent. A total of 25 adjacent events were examined. Thus, NER is intact in G1 synchronous Tax-expressing cells and excision repair of Tax-expressing cells is normal in G1-arrested cells.

Stabilization of p53 and Induction of p21 in Response to UV Exposure in Tax-Expressing Cells

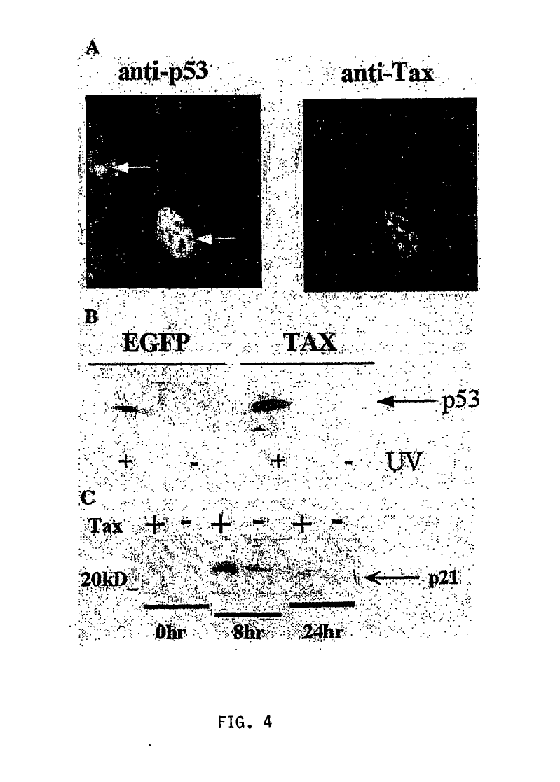

A key player in initiation of the UV-repair response and activation of cell cycle arrest is p53. Activation of p53 is signaled by an accumulation of P53 in the nucleus of cells exposed to UV light. To examine whether Tax-expressing cells display this early p53 response, REF52 cells were transduced with suboptimal viral titers to produce an estimated 50% transduction efficiency. The cells were exposed to UV and examined for nuclear expression of p53. The Tax-expressing REF52 cells displayed a strong nuclear accumulation of P53 in response to UV damage when compared to the same UV-treated REF52 cells not expressing Tax (FIG. 4A). REF52 cells not exposed to UV did not show nuclear accumulation of P53 (data not shown). These results demonstrated that one early step in p53 activation, nuclear accumulation, is intact in Tax-expressing cells.

To further analyze the p53 response in Tax-expressing cells, both Tax-expressing and control REF52 cells were examined for stabilization of steady-state p⁵³ and transient induction of p21. Since induction of p21 is a key event in p53-mediated cell cycle arrest, analysis of the p21 levels in Tax-expressing cells could determine the integrity of this signal. Tax-expressing and control cells were exposed to UV and harvested for western analysis pre-exposure and at eight hours and twenty four hours post exposure. In response to UV-induced DNA damage, both cell groups demonstrated stabilization of steady-state protein levels of p53 (FIG. 4B). Examination of the same cells for p21 expression revealed similar p53-induced transient p21 induction between Tax-expressing and control cells (FIG. 4C). Thus, one p53-dependent signal for cell cycle arrest, transient induction of p21, was intact.

Attenuation of G1 Checkpoint in p53−/− Tax-Expressing Cells

The results from the above experiments suggested that the deficiency in the repair capacity of Tax-expressing cells is due to a failure in cell cycle checkpoint and that this defect is independent of p53 signaling. To test this hypothesis, the cell cycle arrest response was examined in Tax-expressing and control p53−/− cells. Tax-expressing and control Hela cell groups were subjected to the cytokinesis block cell cycle progression assay as described above. Each cell group was exposed to UV and incubated with cytochalasin B one hour later. The cells were examined after 36 hours for the presence of binucleated dividing cells. Each cell group was exposed to UV and incubated with cytochalasin B one hour later. The cells were examined after 36 hours for the presence of binucleated dividing cells. Hela cells transduced to express Tax at 25% efficiency. Tax-expressing Hela cells were identified by immunofluorescence using anti-Tax mouse monoclonal antibody and FITC-conjugated anti-mouse secondary antibody. The nuclei were stained with propidium iodide. Hela and Tax-expressing Hela formed binuclei indicating normal division. When exposed to UV, Hela cells arrested as mononucleated, whereas Tax-expressing cells continued to divide and formed binucleated cells. Similar results were obtained with the p53 deleted cell line. A population of cells was transduced to express Tax at 100% efficiency. Tax expressing p53d cells failed to arrest in response to UV and form binucleated cells. In the experiments using partial transductions of Tax, 100 pairs of adjacent Tax expressing and non-expressing cells were examined. In experiments using 100% transduction of Tax, 1000 cell events were counted. The percent of binucleated control cells was <15% and the percent of binucleated Tax expressing cells was >72% and did not differ significantly between the two approaches.

The results clearly showed that Tax-expressing cells in a p53−/− background failed to generate a DNA-damage cell cycle arrest response. This was true for either p53 “functionally repressed” or p53 “deleted” cell lines expressing Tax. Both p53 repressed and p53 deleted cell lines not expressing Tax showed efficient cell cycle arrest as has been reported elsewhere. Thus, the failed cell cycle arrest is not dependent upon the presence of p53 and is evidence of failure in a pS3-independent DNA damage response.

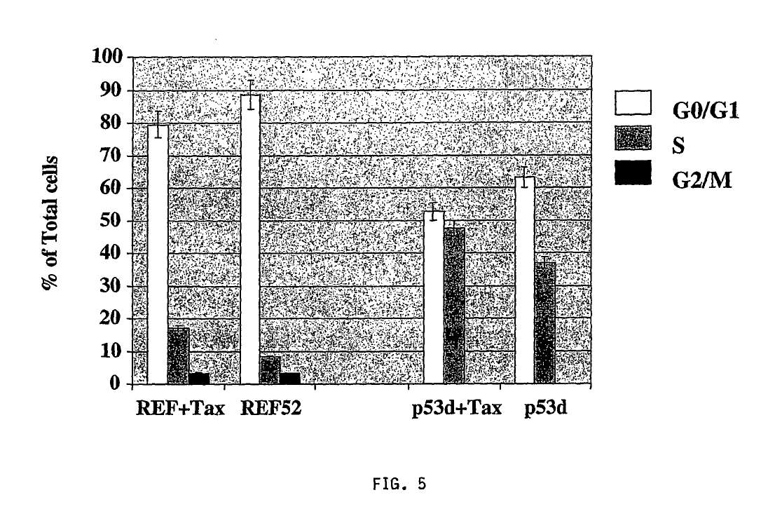

To determine whether the failure in cell cycle arrest was due to a failed G1 checkpoint, cells were synchronized at G0 by serum starvation. The cells were then cultured in 20% serum to stimulate return to cell cycle following UV treatment. Cell cycle was determined by total DNA content as described above. Tax-expressing cells showed a consistent and significant increase in the percentage of cells leaving G1 (FIG. 5). This was true for either a wild type or deleted p53 background. Therefore, the failure in cell cycle arrest is due at least in part to a failed G1 arrest.

Tax-Expressing p53-Cells are Hypersensitive to UV Treatment

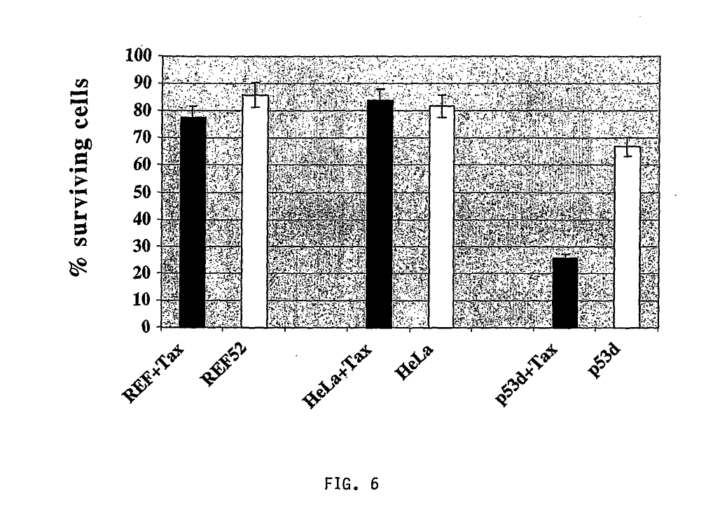

It was reasoned that if Tax-expressing cells display impaired damage response in a p53 independent manner then cells both deleted for p53 and expressing Tax would be more prone to apoptotic cell death in response to UV. To test this hypothesis, the apoptotic response of both Tax-expressing and control p53−/− cells was examined. Tax-expressing and control cells were exposed to sublethal doses of UV and examined for percent surviving cells 24 hours after treatment. Tax-expression in a p53+/+ or p53 mutant background showed no decreased percent of surviving cells over the appropriate control cells. However, Tax-expression in the p53−/− background resulted in significant cell death in response to UV exposure (FIG. 6).

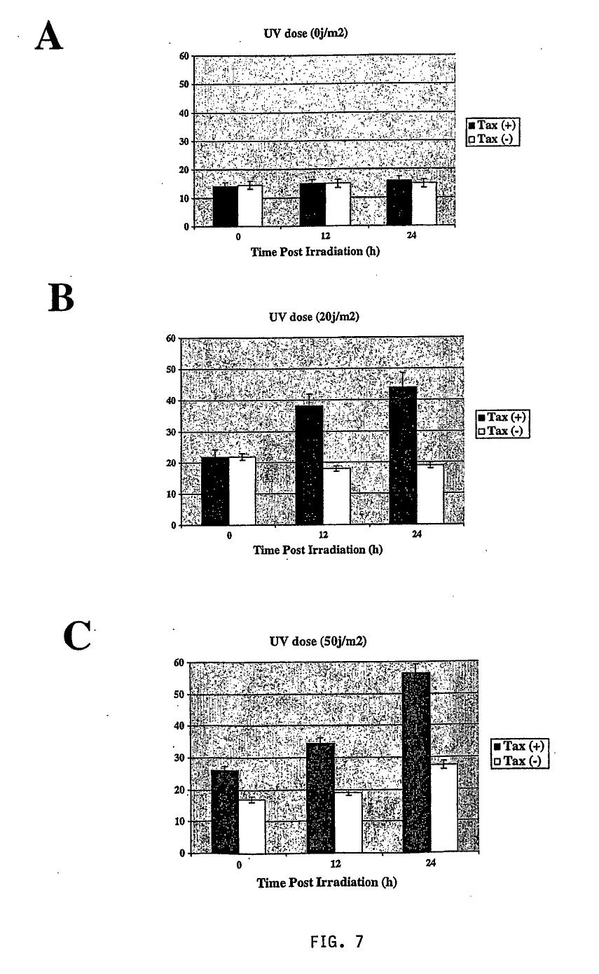

The early apoptotic response of Tax-expressing p53−/− cells was also observed. In this series of experiments the cell groups were exposed to 0 J/m², 20 J/m² and 50 J/m² and apoptosis measured at 12 and 24 hours (FIG. 7). Significant apoptosis was seen in Tax-expressing cells at each time point in response to doses of UV which resulted in only modest induction of apoptosis in control p53−/− cells. This level of UV-induced apoptosis is equivalent to that seen in cells defective in NER (FIG. 3).

Discussion

One of the most compelling hypotheses to explain HTLV-1-mediated cellular transformation is that Tax expression induces genomic instability. In such a model, the viral-induced genomic instability gives rise to increased potential for acquiring discrete genetic changes needed to support leukemogenesis. The initial reports of Tax-induced genomic instability suggested that loss in cellular genomic integrity was the result of the accumulation of genetic damage of both clastogenic and aneuploidogenic nature (Majone, F., et. al., Virology 193(1):456-9 (1993); Semmes, O., et al., Virology 217(1):373-9 (1996)). There is convincing evidence that Tax alone induces a state of genomic instability in the target cell; however, the exact mechanism for this process is unknown.

A mechanistic model for how Tax expression results in increased levels of damaged DNA (clastogenic damage) has centered on a failure in repair-response, which would lead to accumulation of DNA damage. At a molecular level, this has been envisioned to occur by direct transcriptional impairment of repair enzymes such as β-polymerase (Jeang, K. T., et al., Science 247(4946): 10824 (1990)), or as stimulation of PCNA transcription (Ressler, S., et al., J. Virol. 71(2):1181-90 (1997)), implicating defects in both Base-Excision Repair (BER) and Nucleotide-Excision Repair (NER) respectively. Although BER function has not been adequately examined in Tax-expressing cells, the effects of PCNA over expression by Tax, has been linked to reduction in NER activity (Kao, S. Y., et al., J. Virol. 73(5):4299-304 (1999)), as measured in the host-cell reactivation assay (HCRA).

Repression of various tumor suppressor genes has also been suggested to mediate Tax transformation (Yoshida, M., Annu. Rev. Immunol 19:475-496 (2001)), in fact mutation of tumor suppressor genes is common in ATL cells (Hatta, Y., et al., Blood 85(10):2699-704 (1995); Hatta, Y., et al., Br J Haematol. 99(3):665-7 (1997); Nishimura, S., et al., Leukemia 9(4):598-604 (1995); Sakashita, A., et al., Blood 79(2):477-80 (1992)). Central to this discussion is several recent reports implicating impairment of p⁵³ transcription function in the Tax-mediated transformation process (Pise Masison, C. A., et al., Mol Cell Biol. 20(10):3377-86 (2000); Van Orden, K., et al., J Biol. Chem. 274(37):26321-8 (1999)). In one study, over expression of exogenous p53 restored the impaired NER capacity of Tax-expressing cells (Kao, S. Y., et al., Oncogene 19(18):2240-8 (2000)). Implied in these studies is that loss of p53 function would lead to inefficient repair process. However, examination of p53-knockouts have revealed no increase in mutation rate (Griffiths, S. D., et al., Oncogene 14:523 531 (1997)) as would be expected if repair capacity were reduced. In general, early loss of p53 has been associated with improved clonal survival following mutation events. Interestingly, when p53-ATLL were examined there was no evidence of higher mutagenic rates (Takemoto, S., et al., Blood 95:3939-3944 (2000)). However, de novo Tax expression results in increased mutation rates as measured by can resistance in yeast (Semmes, et al., HTLV-1 Tax and loss of genetic integrity. Molecular Pathogenesis of HTLV-1: a current perspective, ed. O. Semmes and M. Hammarskj old. 1999, Arlington: ABI Professional Publishing. 205) and as demonstrated in hrpt resistance in mammalian cells (Miyake, H., et al., Virology 253(2):155-61 (1999)). Therefore, “early” Tax-induced genomic instability, associated with increased mutation frequency, may precede a “late” stage signified by loss in p53 function, which contributes to clonal survival of the newly generated clones. Indeed the two stages are likely interdependent.

In accordance with the present invention, previous observations have been confirmed (Kao, S. Y., et al., J Virol. 73(5):4299-304 (1999)) and it has now been demonstrated that de novo Tax expression resulted in a reduction in cellular NER capacity as measured by the HCRA. The UV-induced repair-response of Tax expressing cells were examined; several key early damage-induced cellular events such as p53 nuclear accumulation and induction of apoptosis were intact. Interestingly, an increased steady-state levels of both p53 and p21 in Tax expressing cells without UV treatment was observed, a result consistent with Tax-induced stabilization of this pathway. However, a notable UV-mediated damage-induced p53 accumulation and transient stabilization of p21 was still evident. Thus, although NER capacity was reduced, the endogenous p53-mediated damage-response events were normal in Tax-expressing cells.

The only functional defect observed in the cellular damage-response was an inability to accommodate cell cycle checkpoint. It has been reported that expression of Tax abrogates G1 arrest induced by exogenous p53 (Mulloy, J. C., et al., J Virol. 72(11):8852-60 (1998)) and subsequently concluded that this result is evidence for alteration of p53 function by Tax. However, induction of G1 arrest by exogenous p53 is dependent on status of endogenous p53 perhaps representing cellular adaptation to loss of p53. Specifically, only p53 null lines demonstrate efficient G1 arrest in response to exogenous p53 (Cascallo, M., et al., Cancer Gene Therapy 6:428-436 (1999); Kagawa, S., et al., Oncogene 15:1903-1909 (1997); Morretti, F., et al., J Clinical Endocrinol Metabolism 85:302-308 (2000); St John, L. S., et al., Cancer Gene Therapy 7:749-756 (2000)), implying that there is a biological difference between damage induced endogenous G1 checkpoint and exogenous p53-induced G1 arrest. It should be noted that damage-induced G1 arrest can occur through p53-independent pathways and indeed we report here attenuation of damage-induced G1 checkpoint in p53 null cells by Tax. These results, therefore, support a mechanistic model in which Tax directly alters cell cycle checkpoint in a manner independent from p53. Synchronizing Tax-expressing cells to G1 resulted in NER capacity that did not differ from normal cells. Thus, checkpoint failure alone could account for the defect in NER capacity. This result allowed for a different interpretation of earlier studies in which exogenous addition of p53 partially restores NER activity of Tax expressing cells. Since exogenously added p53 can induce G1 arrest, rescue of NER by exogenous p53 may result from reinforced G1 arrest and not from restoration of p53 function. Interestingly, a failure in cell cycle arrest in the presence of a competent p21 induction was observed in cells regardless of p53 status. The loss of cell cycle checkpoint in p53−/− cells implied that the effect of Tax is p53 independent and suggested that Tax might affect signals downstream to or independent of p53.

It was speculated that if the Tax-mediated effect were upon a p53-independent pathway then Tax-expressing p53−/− cells would lack both p53-mediated and p53-independent repair responses. A dramatic increase in cell death was observed in response to UV light in Tax-expressing p53−/− cells. The sensitivity to UV in the Tax-expressing p53−/− cells was comparable to that seen in NER-deficient XP-A cells and is in stark contrast to Tax-expressing p53+/+ cells. This result suggested that survival of, and in turn mutational pressure on, Tax-expressing cells is dependent on p53 status and may provide a framework for Tax and p53 interaction. Thus, although the attenuation of UV-mediated cell cycle arrest by Tax does not appear to arise from a direct effect on p53, the status of p53 has profound impact on Tax-expressing cells. Furthermore, since these results were observed following de novo expression of Tax, it was concluded that loss of p53 is selected against in Tax expressing naive cells. The hypersensitivity due to the loss of p53 in de novo Tax-expressing cells may help explain the long period between HTLV-1 infection and cellular transformation. In fact Kao et al., (Kao, S. Y., et al., Virology 291:292-298 (2001)) have shown that stable Tax-expressing p53 null cells display a moderate apoptotic response, suggesting that Tax-expressing cells can adapt to survive p53 loss. Selection against loss of the tumor suppressor p53, which is frequently associated with the phenotype of HTLV-1 transformed cells, would prolong the pre-transformed state of HTLV-1 infected cells. This might explain why Tax has evolved a mechanism for interacting with cellular p53 pathways and why close to 50% of ATL patients present p53 mutations.

The retroviral transduction system of the present invention allowed for examination of de novo expression of Tax and therefore reflected early cellular responses to Tax expression. Events occurring in long-term or stable Tax expressing systems could represent adaptations of the cell to Tax expression and may reflect long-term Tax effects. It was therefore proposed based on these studies that an impairment of the cellular damage-repair response through bypass of appropriate cell cycle checkpoint is a mechanistic route to Tax-induced cellular genomic instability. Moreover, it has been demonstrated that loss of p53 function severely impacts on the survival of Tax-expressing cells, a result that provides an explanation for the inter-dependence between Tax activity and p53 function.

All references cited herein are indicative of the level of skill in the art and are hereby incorporated by reference in their entirety.

1. A method of reducing viability of a targeted p53 null cell comprising introducing into the targeted cell a nucleic acid encoding a polypeptide having human T-cell leukemia virus type I Tax activity, expressing the polypeptide in an effective amount in the targeted cell to thereby enhance sensitivity of the targeted cell expressing said polypeptide to a DNA damaging agent, and administering a DNA damaging agent to the targeted cell expressing the polypeptide thereby reducing viability of the cell.

2. The method of claim 1, wherein the targeted p53 null cell is a cancer cell.

3. The method of claim 1, wherein the DNA damaging agent is a chemotherapeutic agent.

4. The method of claim 3, wherein the chemotherapeutic agent is selected from the group consisting of etoposide, adriamycin, amsacrine, actinomycin D, VP16, camptothecin, colchicine, taxol, cisplatinum, vincristine, vinblastine, and methotrexate.

5. The method of claim 1, wherein the DNA damaging agent is irradiation.

6. The method of claim 5, wherein the irradiation is delivered by exposing the cells to gamma rays, X-rays, directed delivery of radioisotopes, microwaves, or UV radiation.

7. The method of claim 1, wherein the nucleic acid is introduced into the tumor cell by contacting the cell with a retroviral vector containing the nucleic acid.

8. The method of claim 7, wherein the retroviral vector is a lentiviral vector.

9. The method of claim 1, further comprising isolating the targeted cell from a patient prior to introduction of the nucleic acid encoding the Tax polypeptide.

10. The method of claim 9, further comprising the step of reintroducing the targeted cells expressing the Tax polypeptide into the patient prior to contact with the DNA damaging agent.

11. The method of claim 1, wherein the targeted cell is present in a patient.

12. The method of claim 11, wherein the nucleic acid encoding the Tax polypeptide is introduced to a tumor site, wherein the targeted cells are present in the patient.

13. A method for enhancing susceptibility of a patient to DNA damaging agents comprising introducing into a targeted p53 null cell of the patient a nucleic acid encoding a polypeptide having human T-cell leukemia virus type I Tax activity, expressing the polypeptide in an effective amount in the targeted cell to thereby enhance susceptibility of the targeted cell expressing said polypeptide to a DNA damaging agent, and administering the DNA damaging agent to the patient.

14. The method of claim 13, wherein the targeted p53 null cell is a cancer cell.

15. The method of claim 13, wherein the DNA damaging agent is a chemotherapeutic agent selected from the group consisting of etoposide, adriamycin, amsacrine, actinomycin D, VP16, camptothecin, colchicine, taxol, cisplatinum, vincristine, vinblastine, and methotrexate.

16. The method of claim 13, wherein the DNA damaging agent is irradiation.

17. The method of claim 16, wherein the irradiation is delivered by exposing the cells to gamma rays, X-rays, directed delivery of radioisotopes, microwaves, or UV radiation.

18. The method of claim 13, wherein the nucleic acid is introduced into the tumor cell by contacting the targeted cell with a retroviral vector containing the nucleic acid.

19. The method of claim 18, wherein the retroviral vector is a lentiviral vector.

20. The method of claim 13, further comprising isolating the targeted cell from the patient prior to introduction of the nucleic acid encoding the Tax polypeptide into the targeted cell.

21. The method of claim 20, further comprising the step of reintroducing the targeted cells expressing the Tax polypeptide into the patient prior to administration of the DNA damaging agent.

22. The method of claim 13, wherein the nucleic acid encoding the Tax polypeptide is introduced to a tumor site of the patient, wherein the targeted cells are present in the patient.

23. A method of inactivating a tumor in a patient in need thereof comprising introducing into a patient's tumor cells a nucleic acid encoding a polypeptide having human T-cell leukemia virus type I Tax activity, expressing said polypeptide in an effective amount in said tumor cells, thereby enhancing sensitivity of said tumor to a DNA damaging agent, and contacting said tumor with said DNA damaging agent thereby inactivating the tumor.

24. The method of claim 23, wherein the DNA damaging agent is a chemotherapeutic agent selected from the group consisting of etoposide, adriamycin, amsacrine, actinomycin D, VP16, camptothecin, colchicine, taxol, cisplatinum, vincristine, vinblastine, and methotrexate.

25. The method of claim 23, wherein the DNA damaging agent is irradiation.

26. The method of claim 25, wherein the irradiation is delivered by exposing the cells to gamma rays, X-rays, directed delivery of radioisotopes, microwaves, or UV radiation.

27. A method of inducing cell death of a targeted p53 null cell in a patient comprising introducing into the targeted p53 null cell a nucleic acid encoding a polypeptide having human T-cell leukemia virus type I Tax activity, expressing said polypeptide in an effective amount in said targeted p53 null cells, thereby enhancing sensitivity of said cells to a DNA damaging agent, and administering to said cells a DNA damaging agent.

28. The method of claim 27, wherein the DNA damaging agent is a chemotherapeutic agent selected from the group consisting of etoposide, adriamycin, amsacrine, actinomycin D, VP16, camptothecin, colchicine, taxol, cisplatinum, vincristine, vinblastine, and methotrexate.

29. The method of claim 27, wherein the DNA damaging agent is irradiation.

30. The method of claim 29, wherein the irradiation is delivered by exposing the cells to gamma rays, X-rays, directed delivery of radioisotopes, microwaves, or UV radiation.

31. A method for the selective killing of p53 null cells comprising introducing into the targeted p53 null cell a nucleic acid encoding a polypeptide having human T-cell leukemia virus type I Tax activity, expressing said polypeptide in an effective amount in said targeted p53 null cells to enhance sensitivity of said cells to a DNA damaging agent, and administering to said cells a DNA damaging agent, wherein said p53 null cells are selectively killed.

32. The method of claim 31, wherein the DNA damaging agent is a chemotherapeutic agent selected from the group consisting of etoposide, adriamycin, amsacrine, actinomycin D, VP16, camptothecin, colchicine, taxol, cisplatinum, vincristine, vinblastine, and methotrexate.

33. The method of claim 31, wherein the DNA damaging agent is irradiation.

34. The method of claim 33, wherein the irradiation is delivered by exposing the cells to gamma rays, X-rays, directed delivery of radioisotopes, microwaves, or UV radiation.

35. A method for inducing cell death in p53 null cancer cells comprising transducing a p53 null cancer cells with a retroviral vector comprising the nucleic acid encoding the human T-cell leukemia virus type 1 Tax protein, expressing said Tax protein in an effective amount in to sensitize said transduced cells to a DNA damaging agent, and administering to said transduced cells a DNA damaging agent thereby inducing cell death of the transduced cells.

36. The method of claim 35, wherein the DNA damaging agent is a chemotherapeutic agent.

37. The method of claim 36, wherein the chemotherapeutic agent is selected from the group consisting of etoposide, adriamycin, amsacrine, actinomycin D, VP16, camptothecin, colchicine, taxol, cisplatinum, vincristine, vinblastine, and methotrexate.

38. The method of claim 35, wherein the DNA damaging agent is irradiation.

39. The method of claim 38, wherein the irradiation is delivered by exposing the cells to gamma rays, X-rays, directed delivery of radioisotopes, microwaves, or UV radiation.

40. The method of claim 35, further comprising isolating the p53 null cancer cell from a patient prior to introduction of the nucleic acid encoding the Tax protein.

41. The method of claim 40, further comprising the step of reintroducing the targeted cells expressing the Tax protein into the patient prior to contact with the DNA damaging agent.

42. The method of claim 35, wherein the targeted cell is present in a patient.

43. The method of claim 42, wherein the nucleic acid encoding the Tax protein is introduced to a tumor site, wherein the p53 null cancer cells are present in the patient.

44. The method of claim 1, wherein the retroviral vector is a lentiviral vector.
